(19) United States
(12) Patent Application Publication
(10) Pub. No.: US 2020/0315819 A1
(43) Pub. Date: Oct. 8, 2020

(54) Passive Upper Extremity Prosthesis

(71) Applicant: Kimara Belcher, Liverpool, NY (US)

(72) Inventor: Kimara Belcher, Liverpool, NY (US)

(21) Appl. No.: 16/374,338

(22) Filed: Apr. 3, 2019

(51) Int. Cl. A61F 2/80; A61F 2/78; A63D 15/08

(52) U.S. Cl. CPC A61F 2/80; A61F 2/7812; A63D 15/08; A61F 2002/546; A61F 2002/7881; A61F 2002/7818; A61F 2002/7862

(57) ABSTRACT

A passive upper extremity prosthesis for enabling a user with a residual arm to play a cue sport includes a socket that is configured to position over and to couple to a residual arm of a user. A connector that is coupled to the socket is configured to selectively couple to a cue stick to couple the socket to the cue stick, positioning the user to manipulate the cue stick to play a cue sport, such as pool and billiards.

### CROSS-REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM

### Not Applicable

### STATEMENT REGARDING PRIOR DISCLOSURES BY THE INVENTOR OR JOINT INVENTOR

Not Applicable

### BACKGROUND OF THE INVENTION

### (1) Field of the Invention

[symbol]

### (2) Description of Related Art Including Information Disclosed Under 37 CFR 1.97 and 1.98

The disclosure and prior art relates to prostheses and more particularly pertains to a new prosthesis for enabling a user with a residual arm to play a cue sport.

### BRIEF SUMMARY OF THE INVENTION

An embodiment of the disclosure meets the needs presented above by generally comprising a socket that is configured to position over and to couple to a residual arm of a user. A connector that is coupled to the socket is configured to selectively couple to a cue stick to couple the socket to the cue stick, positioning the user to manipulate the cue stick to play a cue sport, such as pool and billiards.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is an isometric perspective view of a passive upper extremity prosthesis according to an embodiment of the disclosure.

FIG. 2 is a bottom view of an embodiment of the disclosure.

FIG. 3 is a side view of an embodiment of the disclosure.

FIG. 4 is a front view of an embodiment of the disclosure.

FIG. 5 is a front view of an embodiment of the disclosure.

FIG. 6 is an in-use view of an embodiment of the disclosure.

### DETAILED DESCRIPTION OF THE INVENTION

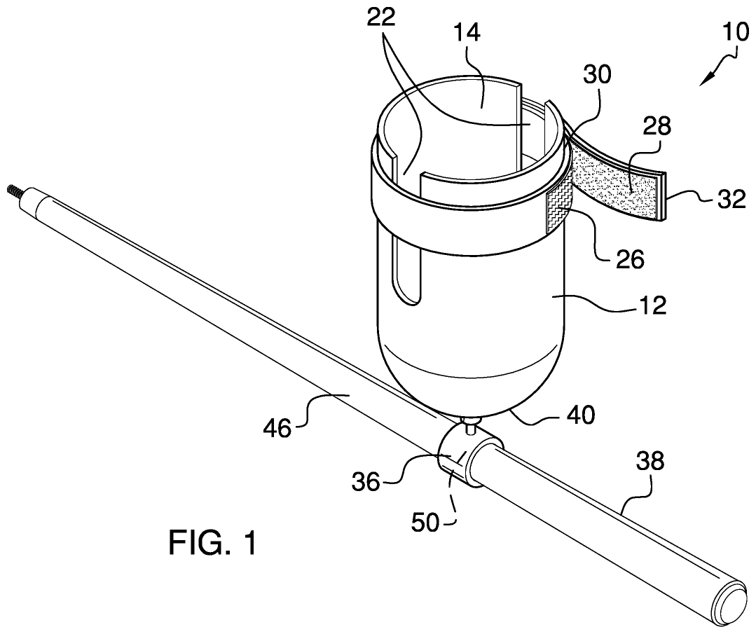

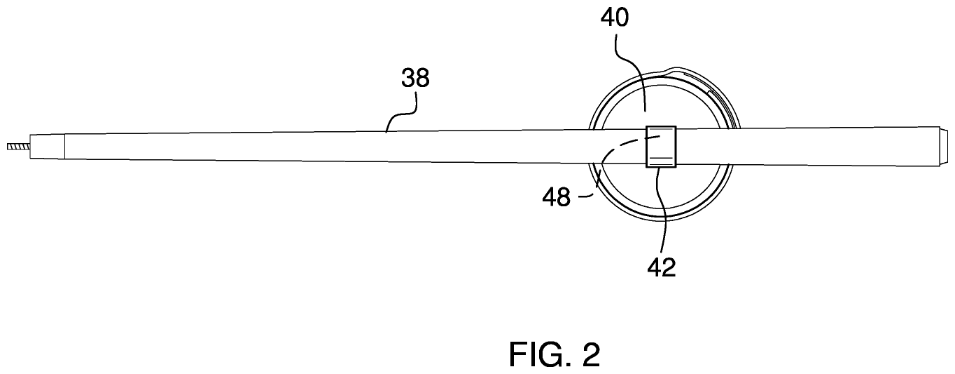

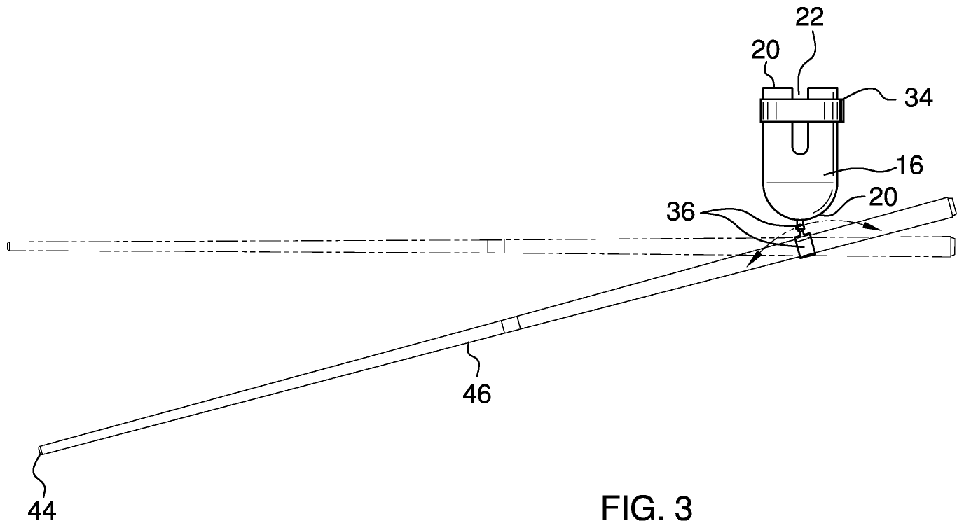

With reference now to the drawings, and in particular to FIGS. 1 through 5 thereof, a new prosthesis embodying the principles and concepts of an embodiment of the disclosure and generally designated by the reference numeral 10 will be described.

As best illustrated in FIGS. 1 through 5, the passive upper extremity prosthesis 10 generally comprises a socket 12 that is configured to position over and to couple to a residual arm of a user. A liner 14, which comprises silicone, may be positioned in the socket 12.

The socket 12 comprises a tube 16 that has a lower end 18 and an upper end 20. The lower end 18 is closed and arcuate so that the lower end 18 is substantially complementary to a profile of an end of the residual arm of the user. The upper end 20 is open and is configured to insert the end of the residual arm of the user to position the tube 16 over the residual arm. The present invention also anticipates the tube 16 being custom made to fit the residual arm of the user.

A plurality of cutouts 22 extends into the tube 16 from the upper end 20 toward the lower end 18. The plurality of cutouts 22 comprises two cutouts 22 that are elongated U-shaped and substantially opposingly positioned in the tube 16, as shown in FIG. 1.

The prosthesis 10 also comprises a strap 24. A first fastener 26 and a second fastener 28 are coupled to a first end 30 and a second end 32 of the strap 24, respectively. The second fastener 28 is complementary to the first fastener 26. The strap 24 is configured to position around the tube 16 proximate to the upper end 20 and to be tightened around the tube 16. The cutouts 22 are configured to contract so that the upper end 20 of the tube 16 abuts the residual arm and so that the second fastener 28 is positioned to couple to the first fastener 26 to fixedly position the strap 24 on the tube 16 to couple the tube 16 to the residual arm. The second fastener 28 and the first fastener 26 comprises a hook and loop fastener 34. The first end 30 of the strap 24 is coupled to the tube 16 proximate to a respective cutout 22.

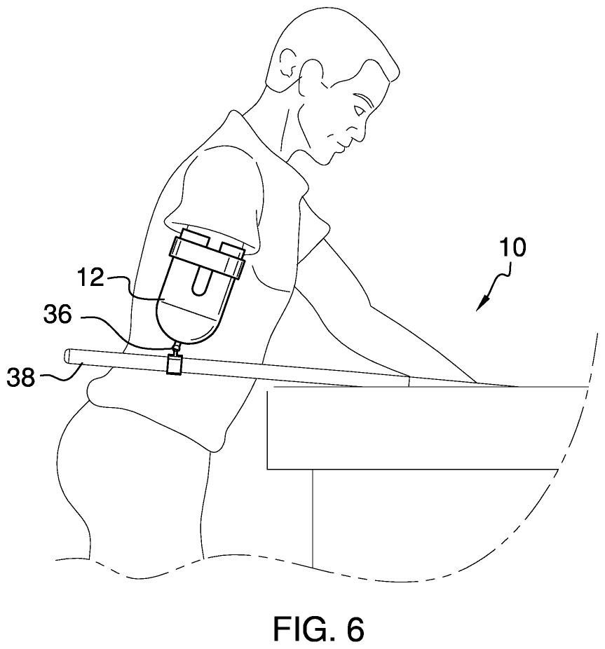

A connector 36 that is coupled to the socket 12, as shown in FIG. 5, is configured to selectively couple to a cue stick 38 to couple the socket 12 to the cue stick 38, positioning the user to manipulate the cue stick 38 to play a cue sport, such as pool and billiards, as shown in FIG. 6. The connector 36 is substantially centrally positioned on a bottom 40 of the socket 12. The present invention anticipates the connector 36 being integral to the socket 12 and the cue stick 38 such that the prosthesis 10 comprises the socket 12, the connector 36, and the cue stick 38. The cue stick 38 also can comprise a bridge stick, not shown, but well known to those skilled in the art of sticks for cue sports.

The connector 36 comprises a ring 42 that is configured to insert a tip end 44 of the cue stick 38 and to slide along a shaft 46 of the cue stick 38 until an inner surface 48 of the ring 42 frictionally couples to the shaft 46 of the cue stick 38 proximate to a natural grasping point 50 on the shaft 46. The inner surface 48 is tapered so that the inner surface 48 substantially matches a profile of the shaft 46 at the natural grasping point 50.

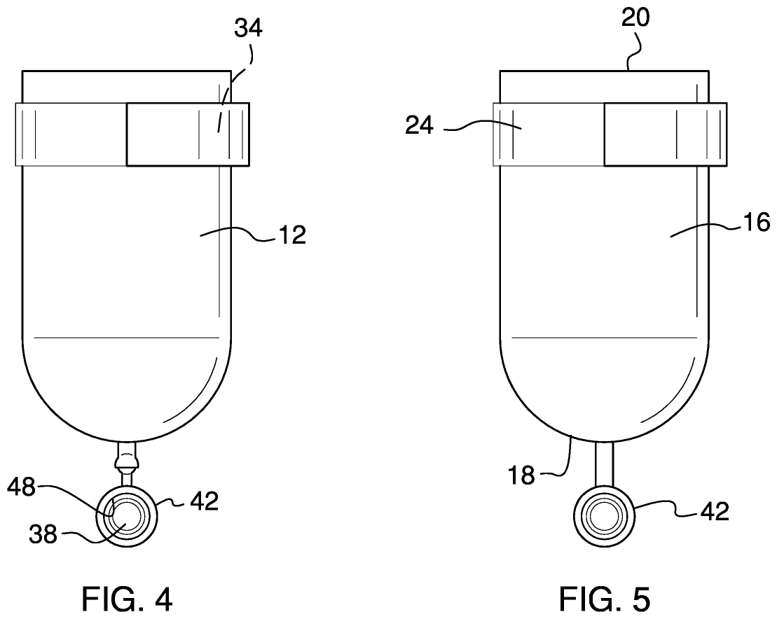

In another embodiment, as shown in FIG. 4, the connector 36 is hingedly coupled to the socket 12 so that the socket 12 is configured to pivot relative to the cue stick 38. In this embodiment, the socket 12 is configured to position over and to couple to an upper arm stump. With the connector 36 pivotally coupled to the socket 12, the pivoting of an elbow can be simulated to allow a user with an upper arm stump to play a cue sport.

In use, the socket 12 is positioned over and coupled to the residual arm of the user. The connector 36 then is coupled to a cue stick 38 to couple the socket 12 to the cue stick 38, positioning the user to manipulate the cue stick 38 to play a cue sport, such as pool and billiards.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. A passive upper extremity prosthesis comprising:
a socket configured for positioning over and coupling to a residual arm of a user; and
a connector coupled to the socket wherein the connector is configured for selectively coupling to a cue stick for coupling the socket to the cue stick positioning the user for manipulating the cue stick for playing a cue sport.

2. The prosthesis of claim 1, further comprising:
the socket being configured for positioning over and coupling to an upper arm stump; and
the connector being hingedly coupled to the socket wherein the socket is configured for pivoting relative to the cue stick.

3. The prosthesis of claim 1, further including the socket comprising:
a tube having a lower end and an upper end, the lower end being closed and arcuate such that the lower end is substantially complementary to a profile of an end of the residual arm of the user, the upper end being open wherein the upper end is configured for inserting the end of the residual arm of the user for positioning the tube over the residual arm;
a plurality of cutouts extending into the tube from the upper end toward the lower end;
a strap;
a first fastener coupled to a first end of the strap; and
a second fastener coupled to a second end of the strap, the second fastener being complementary to the first fastener wherein the strap is configured for positioning around the tube proximate to the upper end and for tightening around the tube wherein the cutouts are configured for contracting such that the upper end of the tube abuts the residual arm and such that the second fastener is positioned for coupling to the first fastener for fixedly positioning the strap on the tube for coupling the tube to the residual arm.

4. The prosthesis of claim 3, further including the cutouts being elongated U-shaped.

5. The prosthesis of claim 3, further including the plurality of cutouts comprising two cutouts being substantially opposingly positioned in the tube.

6. The prosthesis of claim 3, further including the second fastener and the first fastener comprising a hook and loop fastener.

7. The prosthesis of claim 5, further including the first end of the strap being coupled to the tube proximate to a respective cutout.

8. The prosthesis of claim 1, further including a liner positioned in the socket.

9. The prosthesis of claim 8, further including the liner comprising silicone.

10. The prosthesis of claim 1, further including the connector being substantially centrally positioned on a bottom of the socket.

11. The prosthesis of claim 1, further including the connector comprising a ring wherein the ring is configured for inserting a tip end of the cue stick and for sliding along a shaft of the cue stick until an inner surface of the ring frictionally couples to the shaft of the cue stick proximate to a natural grasping point on the shaft.

12. The prosthesis of claim 11, further including the inner surface being tapered such that the inner surface substantially matches a profile of the shaft at the natural grasping point.

13. A passive upper extremity prosthesis and cue stick combination comprising:
a cue stick;
a socket configured for positioning over and coupling to a residual arm of a user; and
a connector coupled to and extending between the socket and the cue stick positioning the user for manipulating the cue stick for playing a cue sport.

14. The prosthesis of claim 13, further comprising:
the socket being configured for positioning over and coupling to an upper arm stump; and
the connector being hingedly coupled to the socket wherein the socket is configured for pivoting relative to the cue stick.

15. A passive upper extremity prosthesis comprising:
a socket configured for positioning over and coupling to a residual arm of a user, the socket comprising:
a tube having a lower end and an upper end, the lower end being closed and arcuate such that the lower end is substantially complementary to a profile of an end of the residual arm of the user, the upper end being open wherein the upper end is configured for inserting the end of the residual arm of the user for positioning the tube over the residual arm,
a plurality of cutouts extending into the tube from the upper end toward the lower end, the plurality of cutouts comprising two cutouts being substantially opposingly positioned in the tube, the cutouts being elongated U-shaped,
a strap,
a first fastener coupled to a first end of the strap, and
a second fastener coupled to a second end of the strap, the second fastener being complementary to the first fastener wherein the strap is configured for positioning around the tube proximate to the upper end and for tightening around the tube wherein the cutouts are configured for contracting such that the upper end of the tube abuts the residual arm and such that the second fastener is positioned for coupling to the first fastener for fixedly positioning the strap on the tube for coupling the tube to the residual arm, the second fastener and the first fastener comprising a hook and loop fastener, the first end of the strap being coupled to the tube proximate to a respective cutout;
a liner positioned in the socket, the liner comprising silicone; and
a connector coupled to the socket wherein the connector is configured for selectively coupling to a cue stick for coupling the socket to the cue stick positioning the user for manipulating the cue stick for playing a cue sport, the connector being substantially centrally positioned on a bottom of the socket, the connector comprising a ring wherein the ring is configured for inserting a tip end of the cue stick and for sliding along a shaft of the cue stick until an inner surface of the ring frictionally couples to the shaft of the cue stick proximate to a natural grasping point on the shaft, the inner surface being tapered such that the inner surface substantially matches a profile of the shaft at the natural grasping point.

16. The prosthesis of claim 15, further comprising:
the socket being configured for positioning over and coupling to an upper arm stump; and
the connector being hingedly coupled to the socket wherein the socket is configured for pivoting relative to the cue stick.
